(19) United States
(12) Patent Application Publication
(10) Pub. No.: US 2016/0015554 A1
(43) Pub. Date: Jan. 21, 2016

(54) COLOSTOMY APPLIANCE

(71) Applicant: Sheik Farouk Khalif GAFOOR, Hong Kong (CN)

(72) Inventor: Sheik Farouk Khalif GAFOOR, Hong Kong (CN)

(21) Appl. No.: 14/333,924

(22) Filed: Jul. 17, 2014

(51) Int. Cl. A61F 5/448

(52) U.S. Cl. CPC A61F 5/448

(57) ABSTRACT

The present invention discloses a colostomy appliance consisting of two pouches, in which one pouch is designed to collect the faecal waste, and the other pouch is designed to receive the above pouch. The first pouch can be easily inserted into the second pouch and removed from the second pouch when it is fully loaded by waste. The liner pouch is made of plastic material which is flushable and biodegradable. The outer pouch is made of laminated breathable PE and nonwoven material, allowing for increased comfort and functionality of collecting waste next to an individual's abdomen. The outer pouch is sprayed on the inside with odour control compound to mitigate the faecal odour. Both the two pouches are designed for ease of use. Wearers can easily carry and replace the inner pouch in public facilities and the materials used for the pouch are silent ensuring discreetness during movement.

### TECHNICAL FIELD

The present invention generally relates to colostomy appliance, and more particularly to a colostomy appliance having removable, disposable, biodegradable and flushable insert liner pouch therein.

### BACKGROUND OF THE INVENTION

The colostomy appliance is commonly used after surgery. But modem colostomy appliance cannot be reused. Further, gas is evolved from the faeces. The accumulation of such gases in colostomy bags, or pouch, causes bloating of the pouch, consequentially results in discomfort and embarrassment to the user. Numerous devices have been developed in an effort to solve the problem. Initially, it is the general practice to provide small openings near the top of such pouches to allow the escaping of gas and heat transfer continuously. While this arrangement could generally avoid discomfort from pressure in the pouch, it nevertheless was not satisfactory as the plastic pouch was not comfortable when in touch with skin.

U.S. Pat. No. 6,015,399 describes a vented colostomy pouch appliance comprising in combination an envelope-like container of thin, flexible waterproof material having a pair of opposite sides and upper and lower portion; a waste aperture adapted to receive waste matter from a wearer; a vent aperture extending through one of said sides at a location in the upper portion of said container; an open-centred generally annular member secured to an exterior surface of said one of said sides in a position generally encircling the vent aperture to form a vent opening; and a plug-type closure member releasably and removably retained within said annular member to seal said vent opening, such that removal of said closure member vents the interior of said container through said opening and replacement of said closure member closed and reseals said opening. This patent solves the ventilation issues using the vent aperture, but reusability and replacement of the pouch are still problems in the technical field.

It results in a higher cost due to the replacement of the used-pouch and the inconvenience of replacing the pouch. Other issues in prior art relate to condensation from body heat and comfort.

Accordingly, colostomy patient continues to experience the basic underlying difficulties and associated problems. Therefore, there is a need to improve the colostomy bag which can be replaced without the spillage of waste, and the removal of bad smell from the bag.

### SUMMARY OF THE INVENTION

This invention responds to the continuing needs referred to above, providing a reliable, effective and removable pouch for ostomy bag. Thus, the primary object of the invention is to provide an improved colostomy appliance including two pouches, in which one pouch is designed to collect faecal waste and the other pouch is designed to receive the above pouch. Specifically, the technical solution of the invention is as follows:
a colostomy appliance comprising:
a first pouch formed by breathable material for securing to a wearer;
a second disposable pouch formed by a plastic material to receive waste matter from the wearer, which can be easily removed and disposed by flushing down any lavatory;
an opening located on one side of the first pouch for inserting the second pouch into the first pouch and removing the second pouch therefrom; and
means for opening and sealing the opening.

In one embodiment, the second pouch is an insert liner pouch, which can be easily inserted into the first pouch.

According to one embodiment of the colostomy appliance of the invention, the plastic material is biodegradable, which is selected from the group consisting of polyethylene or polypropylene, or both.

According to one embodiment of the colostomy appliance of the invention, the breathable material is polyethylene infused non-woven material treated with titanium dioxide to provide specification level of breathability of 2500 grams of water vapour per 24 hour period over one square meter under conditions of 60% relative humidity and 400° C. temperature.

According to another embodiment of the colostomy appliance of the invention, the opening of first pouch is formed by a die-cutting machine, which can be opened and sealed by an adhesive tape provided with the pouch.

According to another embodiment of the colostomy appliance of the invention, a ring is embedded into the other side of the first pouch by heat sealing.

According to one embodiment of the colostomy appliance of the invention, odour control chemical is sprayed on the body of the first pouch and the ring to eliminate the smell of the colostomy contents.

According to another embodiment of the colostomy appliance of the invention, the second pouch has a wide seal at the edge.

According to another embodiment of the colostomy appliance of the invention, ventilation holes are arranged on the second pouch.

According to yet another embodiment of the colostomy appliance of the invention, a colour indicator band for fixing position is provided on the second pouch.

Accordingly, the colostomy appliance of the invention contains two pouches formed by different material to achieve the above object of reusing the colostomy appliance. The outer pouch is secured to the patient and designed for easy insertion and removal of the loaded inner ostomy pouch without spillage of the waste. The outer pouch is made of breathable, liquid resistant material and the inner pouch is made of biodegradable and flushable polymeric film for convenient disposal, while they may be environment friendly. The inner pouch has an indicator band for easy positioning during attachment.

For replacement of the inner pouch, the outer pouch has a closable flap for easy insertion and removal of the inner pouch designed to prevent spillage of the waste during removal.

In addition, both the two pouches are designed for ease of use. Wearer can easily carry and replace the inner pouch in public area. The materials used for the bag are silent ensuring discreetness during movement.

### BRIEF DESCRIPTION OF THE DRAWINGS

The features of the present invention may be clearly set forth with reference to the accompanying drawings. The invention, together with the advantages thereof may be best understood by reference to the following description taken in conjunction with the accompany drawings, wherein like reference signs identify like elements, and wherein:

FIG. 1 is a front schematic view of a first pouch of the colostomy appliance, according to one embodiment of the invention;

FIG. 2 is a rear schematic view of a first pouch of the colostomy appliance, according to one embodiment of the invention; and

FIG. 3 is a schematic view of a second pouch of the colostomy appliance, according to one embodiment of the invention.

### DETAILED DESCRIPTION OF THE INVENTION

The structure of the appliance of the invention will be explained in detail with reference to the accompany drawings. The description and explanatory embodiment herein are merely used to set forth the present invention, not to limit the invention.

In this text, the term “pouch” means all kinds of bags made of different material. “The first pouch” herein is a bag that accommodating the second pouch, where “the second pouch is a bag made of plastic, which is called an “insert liner”. That is to say, the second pouch and the “insert liner” mean substantially the same thing herein.

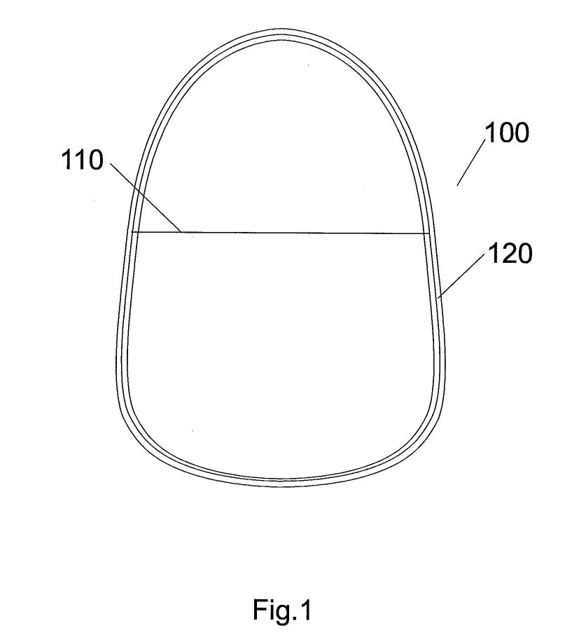

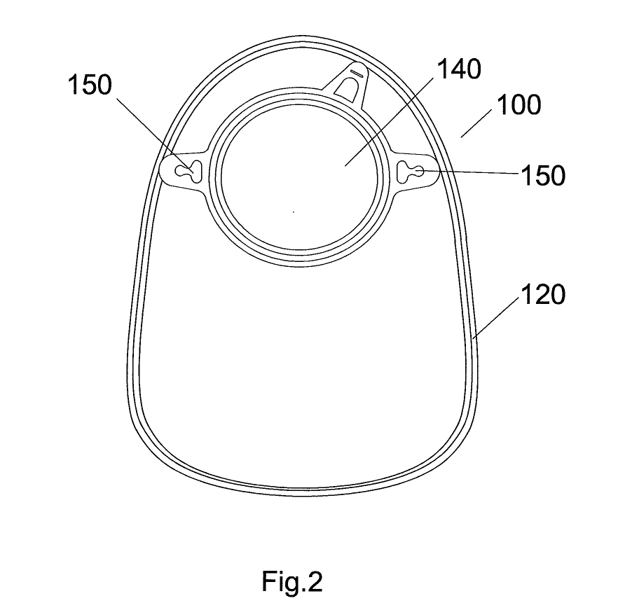

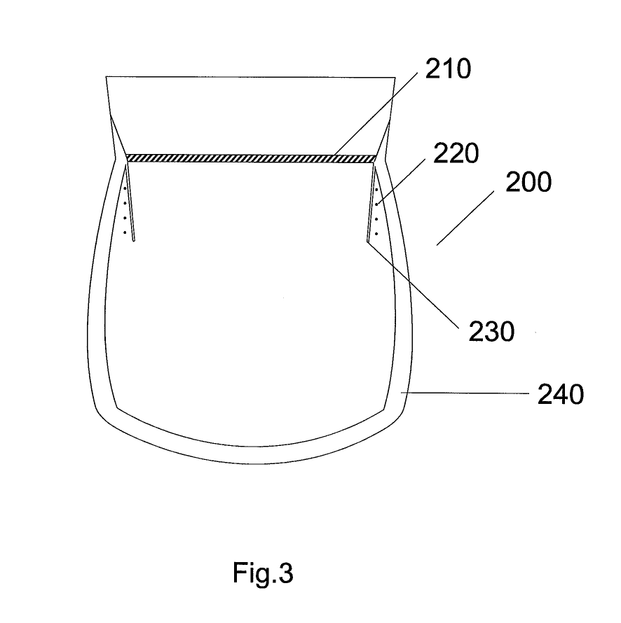

Referring now to the drawings in detail, a colostomy appliance embodying the present invention is showed, which comprises a first pouch 100 and a second pouch 200, and the second pouch 200 can be inserted and disposed into the first pouch 100, when used. Referring to FIGS. 1-3, the second pouch 200 is used to receive faecal waste, and the first pouch 100 is secured to a wearer. Securing the first pouch 100 is a common knowledge in prior art, which is omitted herein.

As conventional, the first pouch 100 is made of breathable material which can help heat and air exchange of the colostomy appliance. Preferably, the said breathable material is polyethylene infused non-woven material. Due to the nature of the polyethylene and the non-woven material, the first pouch 100 contains a plurality of ventilation holes for heat transfer. Thus, better ventilation effect can be obtained.

Preferably, on one side of the first pouch 100, an opening 110 is cut which allows insertion of the second pouch 200 into the first pouch 100, and is also used to remove the loaded second pouch 200 out of the first pouch 100. For closing the opening, means for opening and sealing the opening are provided. Preferably, the means is an adhesive tape fixed on the opening 110. Preferably, the first pouch 100 has a seal 120 to seal this pouch.

The second pouch 200 is formed by plastic material, to receive waste matter from the wearer, which is removably disposed in the first pouch 100. The plastic material is biodegradable, which can be selected from the group consisting of polyethylene or polypropylene, or both. More preferably, biodegradable polypropylene is used to manufacture the second pouch 200. Further, the edge of the second pouch 200 is heat sealed, like reference signs 240, to form a sealing area, which has a width of 0.6-0.8 cm. This seal 240 ensures that the second pouch 200 may not be broken when it is fully loaded by waste. Further, the upper portion of the second pouch 200 is also sealed (see reference sign 230).

The second pouch 200 contains a plurality of ventilation holes 220 for pressure release which is protected by seal 230. And the seal 230 can prevent the waste from leakage. A colour (such as red and yellow) indicator band 210, is located on the neck of the second pouch 200 for fixing position. Preferably, the colour band 210 has a depth of 3 mm and a length of 12 cm to allow a clear identification of the position.

The first pouch 100 contains a ring 140 having 3 ears 150 on the other side for easy operation of the first pouch 100. In order to eliminate the strange odour from the pouch, some odour control chemicals are sprayed on the first pouch 100 by using a nozzle and then left to dry naturally.

The colostomy appliance of the invention can be easily operated, which allows the user to simply replace the inner second pouch 200 when it is loaded, even in public area, while avoiding the spillage of the waste. Thus, replacement of the inner second pouch 200 is more cost effective than replacing the whole appliance by user.

Furthermore, due to the manufacturing material's property, the second pouch 200 is environmental friendly and can keep silent when replacing the pouch.

While a particular embodiment of the invention has been shown and described, it will be obvious to those skilled in the art that changes and modifications can be made without departing from the true spirit and scope of the invention. It should be understood that the embodiments of the present invention described above are illustrative only, and all the changes and modifications made by those skilled in the art are covered by the appended claims.

What is claimed is:

1. A colostomy appliance comprising:
a first pouch formed by breathable material for securing to a wearer;
a second disposable pouch formed by plastic material to receive waste matter from the wearer, which is removably disposed in the first pouch;
an opening located on one side of the first pouch for inserting the second pouch into the first pouch and removing the second pouch therefrom; and
means for opening and sealing the opening.

2. The colostomy appliance according to claim 1, wherein the plastic material is biodegradable and is selected from the group consisting of polyethylene or polypropylene, or both.

3. The colostomy appliance according to claim 1, wherein the breathable material is polyethylene infused non-woven material treated with titanium dioxide to provide specification level of breathability of 2500 grams of water vapour per 24 hour period over one square meter under conditions of 60% relative humidity and 40° C. temperature.

4. The colostomy appliance according to claim 1, wherein the opening of first pouch is formed by a die-cutting machine, which can be opened and sealed by an adhesive tape.

5. The colostomy appliance according to claim 1, wherein a ring is embedded into the other side of the first pouch by heat sealing.

6. The colostomy appliance according to claim 1, wherein odour control chemical is sprayed on the body of the first pouch and the ring to eliminate the smell of the colostomy appliance.

7. The colostomy appliance according to claim 1, wherein the second pouch has a wide seal at the edge.

8. The colostomy appliance according to claim 1, wherein ventilation holes are arranged on the second pouch.

9. The colostomy appliance according to claim 1, wherein a colour indicator band for fixing position is provided on the second pouch.

10. The colostomy appliance according to claim 9, wherein the indicator is red or yellow, and has a depth of 3 mm and a length of 12 cm.
